(19) United States
(12) Patent Application Publication
(10) Pub. No.: US 2009/0018332 A1
(43) Pub. Date: Jan. 15, 2009

(54) Processes For Preparing Bicyclic Oxazine Carboxaldehyde and Beta-Lactamase Inhibitors

(75) Inventors: Kenneth Alfred Martin Kremer, Lawrenceville, NJ (US); Lalitha Krishnan, Suffern, NY (US); Aranapakam Mudumbai Venkatesan, Rego Park, NY (US); Mellard Norman Jennings, Highland Falls, NY (US); Joseph Zeldis, New City, NY (US); Takao Abe, Sakado-shi (JP); Tarek Suhayl Mansour, New City, NY (US); Henry Lee Strong, Somerset, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/144,733

(22) Filed: Jun. 24, 2008

Related U.S. Application Data
(60) Provisional application No. 60/937,612, filed on Jun. 28, 2007.

(51) Int. Cl. C07D 498/04

(52) U.S. Cl. 544/105

(57) ABSTRACT

The invention relates to processes for the preparation of the bicyclic oxazine carboxaldehyde Compound 1:

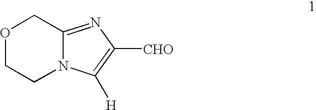

The invention also relates to the use of Compound 1 in the preparation of □-lactamase inhibitors.

This application claims the benefit of U.S. Provisional Application No. 60,937612, filed Jun. 28, 2007, the entire disclosure of which is incorporated by reference herein.

### BACKGROUND OF THE INVENTION

This invention relates to a novel process for the synthesis of bicyclic oxazine carboxaldehydes that are useful in the synthesis of β-lactamase inhibitors.

Bacteria are quickly developing resistance to antibiotics. As a result, mankind constantly races to find new and improved ways to treat bacterial diseases that overcome or circumvent this bacterial resistance. A promising method to improve the efficacy of antibiotics is to inhibit the antibiotic resistance pathways bacteria use to protect themselves, such as the β-lactamase pathway.

β-Lactamases are enzymes produced by bacteria that hydrolyze β-lactam antibiotics and serve as the primary pathway of bacterial resistance to β-lactam antibiotics; such as penicillins and cephalosporins, which are the most widely used β-lactam antibiotics. However, the development of resistance to β-lactam antibiotics by pathogens has hindered the effective treatment of bacterial infections. (Coleman, K., Expert Opin. Invest. Drugs 1995, 4, 693; Sutherland, R., Infection 1995, 23 (4) 191; Bush, K., Cur. Pharm. Design 1999, 5, 839-845). The most significant known mechanism for the development of bacterial resistance to β-lactam antibiotics is the production of class-A, class-B, and class-C serine β-lactamases. These enzymes degrade β-lactam antibiotics, resulting in the loss of antibacterial activity. Class-A enzymes preferentially hydrolyze penicillins while class-C β-lactamases preferentially hydrolyze cephalosporins. (Bush, K., Jacoby, G. A., Medeiros, A. A., Antimicrob. Agents Chemother. 1995, 39, 1211). In fact, over 250 different β-lactamases have been reported to date, many of which have no reported inhibitors (Payne, D. J., Du, W., and Bateson, J. H., Exp. Opin. Invest. Drugs 2000, 247). Bacterial resistance to β-lactam antibiotics may be reduced by administering these antibiotics with a compound that inhibits one or more β-lactamases. Thus, there is a need for a new generation of broad-spectrum β-lactamase inhibitors.

In order to bring β-lactamase inhibitors to the public, a practical process to synthesize these inhibitors must be developed. A critical step towards the practical synthesis of β-lactamase inhibitors is the development of convenient and economical processes to synthesize intermediates that are useful in the synthesis of these inhibitors.

### SUMMARY OF THE INVENTION

In one embodiment, the invention relates to a process for preparing Compound 1:

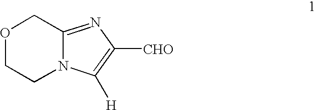

the process comprising the steps of:
reacting Compound 2 or a salt thereof:

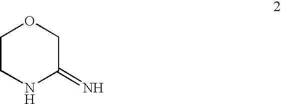

with Compound 3:

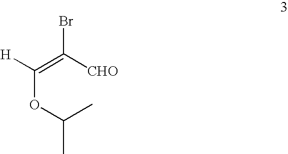

in the presence of a first base, selected from the group consisting of an alkali carbonate and an amine base, to yield Compound 1.

Another embodiment relates to a process for preparing Compound 1, the process comprising the steps of:
reacting Compound 2 or a salt thereof:

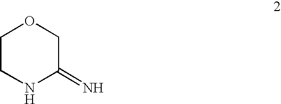

with Compound 10:

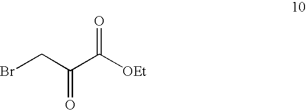

in the presence of an alkali carbonate to yield Compound 11:

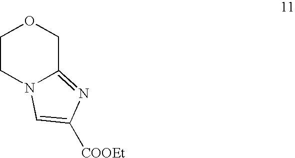

and reducing compound 11 with a reducing agent to yield compound 1.

In some embodiments the invention relates to the use of compound 1 in a process to prepare β-lactamase inhibitors, such as a compound of formula 12:

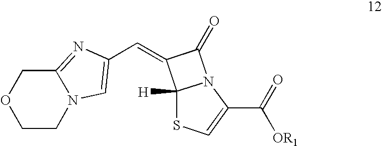

wherein
   R₁ is H, a salt selected from Na, K, and Ca, or an in vivo hydrolyzable ester selected from a C₁₋₆alkyl, a C₅₋₆cycloalkyl, or a —CHR₂OC(O)C₁₋₆alkyl; and
   R₂ is H, a C₁₋₆alkyl, a C₃₋₆cycloalkyl, an optionally substituted C₃₋₁₄aryl, or an optionally substituted heteroaryl,
or a pharmaceutically acceptable salt or hydrate thereof;
the process comprising the steps of:
   condensing Compound 1:

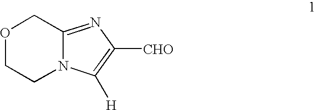

with a 6-bromo-penem derivative of formula 13:

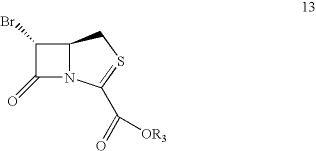

in the presence of a Lewis acid and a fifth base,
wherein R₃ is para-nitrobenzyl, benzyl, para-methoxy benzyl, benzhydrol, or trityl, to form an intermediate aldol product of formula 14:

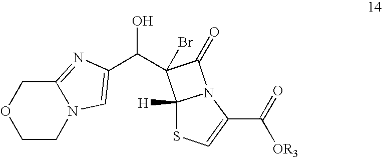

wherein R₃ is as defined above;
reacting the intermediate aldol product of formula 14 with an acid chloride of the formula R₄Cl, an anhydride of the formula (R₄)₂O, or C(X₁)₄ and triphenylphosphine,
wherein R₄ is C₁₋₆alkyl-SO₂—, C₃₋₁₄aryl-SO₂—, C₁₋₆alkyl-C(O)—, or C₃₋₁₄aryl-C(O)—;
and X₁ is Br, I, or Cl,
to form an intermediate compound of formula 15:

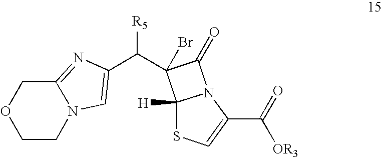

wherein R₅ is —OR₄ or X₁ and R₃, R₄ and X₁ are as defined above; and
   converting the intermediate compound of formula 15 to the compound of formula 12, or a pharmaceutically acceptable salt or hydrate thereof, by a reductive elimination process.

Some embodiments relate to Compound 1:

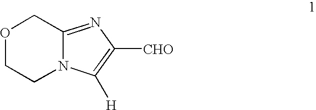

or a salt or hydrate thereof.

Other embodiments relate to Compound 2:

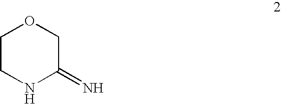

or a salt or hydrate thereof.

In other embodiments the invention relates to Compound 3:

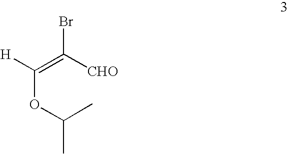

or a salt or hydrate thereof.

Other embodiments relate to a compound 6:

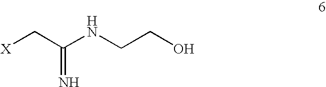

wherein X is Cl, Br, or I,
or a salt or hydrate thereof.

Other embodiments the invention relates to Compound 8:

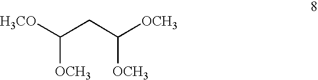

or a salt or hydrate thereof.

Other embodiments of the invention relate to Compound 9:

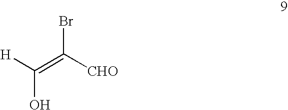

or a sa t or hydrate thereof.

Other embodiments the invention relate to Compound 10:

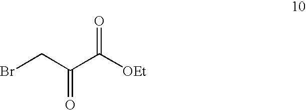

or a salt or hydrate thereof.

Other embodiments relate to Compound 1:

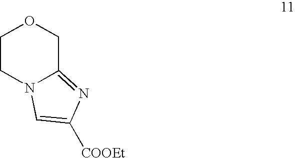

or a salt or hydrate thereof.

Other embodiments relate to a compound of formula 14:

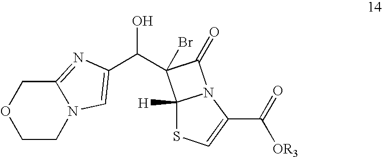

wherein R₃ is para-nitrobenzyl, benzyl, para-methoxy benzyl, benzhydrol, or trityl, or a salt or hydrate thereof.

Other embodiments relate to a compound of formula 15:

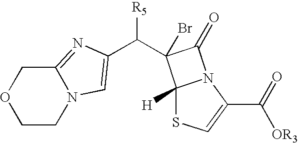

wherein:

R₃ is para-nitrobenzyl, benzyl, para-methoxy benzyl, benzhydrol, or trityl; and

R₅ is —OR₄ or X₁;

R₄ is C₁-C₆ alkyl-SO₂—, C₃₋₁₄aryl-SO₂—, C₁-C₆ alkyl-C(O)—, or C₃₋₁₄aryl-C(O)—; and

X₁ is Br, I, or Cl,

or a salt or hydrate thereof.

Other embodiments relate to Compound 16:

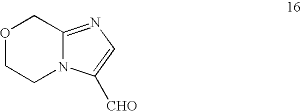

or a salt or hydrate thereof.

Other embodiments relate to Compound 18:

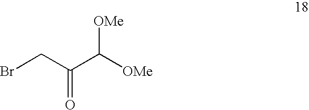

or a salt or hydrate thereof.

### DETAILED DESCRIPTION OF THE INVENTION

In one aspect, the invention relates to processes for preparing 5,6-dihydro-8H-imidazo[2,1-c][1,4]oxazine-2-carbaldehyde, Compound 1.

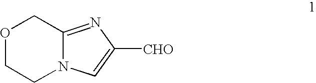

In one embodiment, Compound 1 may be prepared by
reacting Compound 2 or a salt thereof:

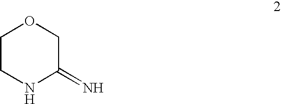

with Compound 3:

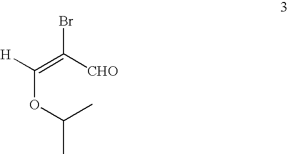

in the presence of a first base, selected from an alkali carbonate and an amine base, to yield Compound 1.

The process may be carried out in an organic solvent, for example an organic solvent selected from acetone, N,N-DMAc, THF, ethyl acetate, ethyleneglycol diethyl ether, 1,2-dimethoxyethane, 1,2-dichloroethane, NMP, DMF, acetonitrile, DMSO, toluene, sulfolane, and ethanol. Other organic solvents are described below.

The amine base may for example be selected from 4-methylmorpholine, triethylamine, 2,6-lutidine, 2,2,6,6-tetramethylpiperidine, N,N′-diethylaniline, DBN, pyridine, diethylamine, and ethanolamine. Other amine bases are described below.

The salt of Compound 2 may be, for example, an acetate salt or a hydrochloride salt.

In another embodiment, Compound 1 may be prepared by reacting Compound 2 or a salt thereof:

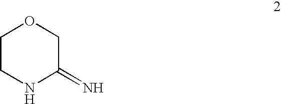

with Compound 10:

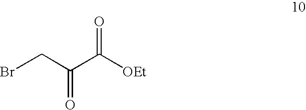

in the presence of an alkali carbonate to yield Compound 11

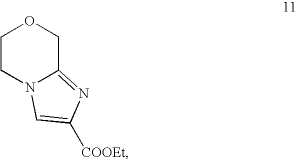

and reducing Compound 11 with a reducing agent to yield Compound 1.

The process may be carried out, for example, in an organic solvent; for example, dimethoxyethane may be employed.

Preferred embodiments and steps of the processes are described in more detail, below.

In another aspect, the invention relates to the use of Compound 1 in processes to prepare β-lactamase inhibitors. The steps of the processes are described in more detail, below.

U.S. Pat. No. 7,112,582 and U.S. Patent Publication Nos. 2004/0132708, 2004/0053913, and 2006/0217361 disclose some methods to synthesize some β-lactamase inhibitors and their intermediates, each of which is hereby incorporated by reference in its entirety. In some aspects of the invention, the ease of making Compound 1, some β-lactamase inhibitors, and their intermediates can be enhanced relative to earlier methods.

An example of a process to prepare 5,6-dihydro-8H-imidazo[2,1-c][1,4]oxazine-2-carbaldehyde, Compound 1 is shown in Scheme 1. In Scheme 1 suitable reaction conditions are shown, however, other reaction conditions may be used within the scope of the invention. For example shorter or longer reaction times may be employed; generally the longer the reaction time, the more complete the reaction; and other bases and organic solvents may be employed.

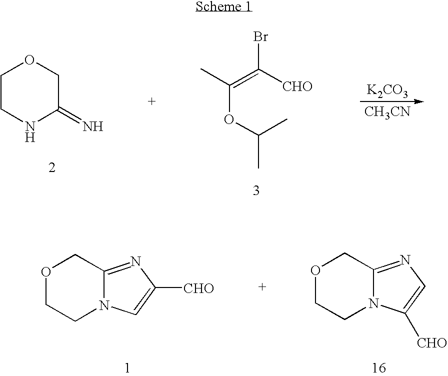

Scheme 1 shows an embodiment wherein Compound 1 can be synthesized by coupling a morpholin-3-ylideneamine, such as (2) or a salt thereof such as a hydrochloride or acetate salt, and an activated compound such as 2-bromo-3-isopropoxy-propenal, Compound 3. Specifically, morpholin-3-ylideneamine (2) can be annulated with 2-bromo-3-isopropoxy-propenal (3) under anhydrous, basic conditions, in the presence of a base such as anhydrous potassium carbonate (K₂CO₃), in an anhydrous organic solvent, such as anhydrous acetonitrile or THF, to yield a bicyclic oxazine carboxaldehyde, such as Compound 1, or a mixture of both Compound 1 and Compound 16. In one aspect, Compound 3, or an anhydrous solution thereof, can be added slowly to an anhydrous solution of Compound 2, at about room temperature such as about 20° C., and anhydrous potassium carbonate added. The mixture can be heated, such as to about 70° C. In one aspect, the mixture can be heated for from about 15 to about 30 minutes, then cooled to a temperature from about 20° C. to about 30° C., or to about room temperature. Compound 1 can then be isolated from the reaction mixture.

In one aspect, Compound 1 can be purified from the reaction mixture using crystallization. For example, the base can be removed from the reaction mixture at about room temperature, such as between about 20° C. and about 35° C., such as by filtering the mixture to remove a solid base. The reaction filtrate is then washed with an organic solvent such as acetonitrile. The combined filtrate and wash can be concentrated, and the concentrate partitioned between water or a brine solution and a water-immiscible organic solvent such as methylene chloride (DCM). The DCM can then be separated, the aqueous layer extracted with more portions of DCM, and the DCM portions combined and concentrated until crystallization begins. An organic solvent such as tert-butyl methyl ether (TBME) can then be added to enhance the crystallization of Compound 1. The crystallization mixture can then be concentrated, and more TBME added to further increase the crystallization of Compound 1 while retaining Compound 16 primarily in the residual DCM. The TBME addition and subsequent concentration procedure can be repeated until no more crystals form or the residual oil no longer decreases in viscosity, as judged visually or by methods known to one of skill in the art. The crystals of Compound 1 can be filtered off, washed if desired, and dried. Alternatively, the combined DCM portions from the aqueous partitioning and washing can be evaporated. Then minimal DCM can be added and the TBME addition and concentration procedure detailed above can be followed from thereon, yielding crystals of Compound 1.

In another embodiment, Compound 2 or a salt thereof, such as a hydrochloride or an acetate salt (Compound 17, below), can be added slowly to a basic mixture of Compound 3, such as in the presence of anhydrous potassium carbonate in an anhydrous organic solvent such as anhydrous acetonitrile. The slow addition of Compound 2 or its salt to Compound 3 can result in an enhanced yield and regioselectivity of formation of Compound 1.

In one embodiment, Compound 10 may be used in place of Compound 3, such as is shown in Scheme 2. In Scheme 2 suitable reaction conditions are shown, however, other reaction conditions may be used within the scope of the invention. For example shorter or longer reaction times may be employed; generally the longer the reaction time, the more complete the reaction; and other bases and organic solvents may be employed.

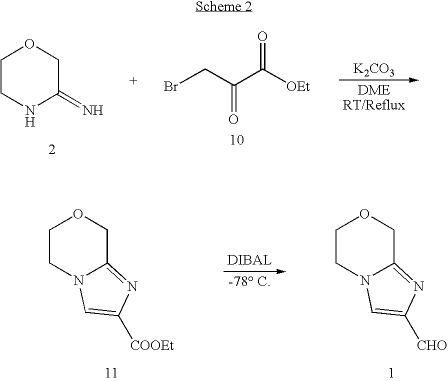

In another embodiment, shown in Scheme 2, Compound 1 can be synthesized by coupling a morpholin-3-ylideneamine, such as 2 or a salt thereof, such as a hydrochloride or acetate salt, and an activated compound, such as Compound 10, under basic conditions such as via an intermediate ester (11) that can be reduced to Compound 1. For example, a hydrochloride salt of Compound 2 in an anhydrous, polar organic solvent, such as an anhydrous ethylene glycol ether, such as dimethoxyethane (DME), can be reacted with ethyl-bromopyruvate (10) in the presence of a base, such as anhydrous potassium carbonate, at room temperature or above, such as at reflux, for a time such as 16 hours, to form Compound 11. In one aspect, Compound 11 can be isolated from the reaction mixture. For example, the reaction mixture can be concentrating and extracted with a water-immiscible organic solvent such as chloroform, which may then be dried, such as over anhydrous Na₂SO₄, filtered, and concentrated. The crude product can be purified by silica (SiO₂) column chromatography, such as by eluting with 1:1:0.05 ethylacetate:hexane:methanol.

In yet another embodiment, shown in Scheme 2, the ester (11) can be dissolved in an anhydrous organic solvent such as anhydrous THF and cooled to a low temperature, such as below about 0° C., or below about −40° C., or to about −78° C., and a reductant such as Diisobutylaluminum hydride (DIBAL) slowly added. The reaction can be stirred, such as for 2 hours, while the temperature is slowly elevated to −40° C., then further stirred at about 40° C. for about another hour. The reaction mixture can then be quenched, for example with a solution of ammonium chloride. In one aspect, Compound 1 can then be isolated. For example, the quenched reaction mixture can be extracted with a water-immiscible organic solvent such as chloroform. The extract can be washed with a saturated salt solution, such as sodium chloride (brine), then dried, such as over anhydrous Na₂SO₄, filtered, and concentrated. The concentrate can be purified by SiO₂ column chromatography, such as by eluting with ethylaceate:hexane (4:1), to yield purified Compound 1.

In one aspect, the use of Compound 10 in place of Compound 3 can enhance the regioselectivity of formation of Compound 1 instead of its regioisomer, Compound 16. Compound 3, however, has enhanced shelf life over Compound 10.

In other embodiments, an activated pyruvaldehyde or activated pyruvaldehyde-diacetal, such as the dimethylacetal, Compound 18, can be used in place of Compound 3 in Scheme 1 or Compound 10 in Scheme 2:

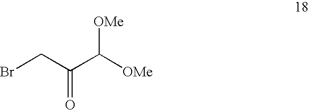

The activated pyruvaldehyde-diacetal can be any common diacetal such as C₁₋₆alkyl diacetals such as dimethyl, diethyl, or diisopropyl acetals, or cyclic acetals such as acetonide.

In one embodiment, organic solvents used in Scheme 1 or 2 can be anhydrous, such as anhydrous acetonitrile, anhydrous tetrahydrofuran, or an anhydrous ethylene glycol ether. Other organic solvents that may be used in Scheme 1 or Scheme 2 include ketones such as acetone; N,N-dimethyl acetamide (N,N-DMAc); N,N-dimethylformamide (DMF); THF; acetates such as methyl, ethyl, or propyl acetates; ethylene glycol ethers such as ethylene glycol diethyl ether or 1,2-dimethoxyethane (DME); chlorinated organic solvents such as methylene chloride (DCM), chloroform, or 1,2-dichloroethane (DCE); N-methylpyrrolidone (NMP); acetonitrile or propionitrile; dimethyl sulfoxide (DMSO); toluene; a sulfolane; alcohols such as methanol, ethanol, or propanol such as iso-propanol; hexane; heptane; cyclohexane; and methyl tert-butyl ether. Organic solvents such as those above may be used in reactions depicted in the schemes herein as will be apparent to one of skill in the art.

The base used in the basic conditions for converting Compounds 2 or its salt and Compounds 3, 10, or 18 to Compound 1 and/or 16 can be present in at least a stoichiometric amount, to soak up acid generated in the reaction, such as HBr. In other embodiments, an excess of base can be used. In yet other embodiments, no base need be used.

In one aspect, the base used in the basic conditions of any step herein can be an alkali carbonate, such as lithium, sodium, potassium, cesium, calcium, or magnesium carbonate, or a base having a similar pKa. Alternatively, other bases can be used, including organic, inorganic, phosphazene, or solid-phase resin bases, and the bases can be liquids or solids. Other bases can include other alkali bases, such as alkali alkoxides, oxides, or hydroxides, with the proviso that a base used in Scheme 1 or Scheme 2 is not potassium t-butoxide. In one embodiment, the base used in Scheme 1 or Scheme 2 is not an alkali alkoxide. Alkali bases can include lithium, sodium, potassium, cesium, calcium, or magnesium salts of alkoxides (such as methoxide or t-butoxide), oxides, or hydroxides. Amine bases include pyridine or pyridine derivatives including 4-dimethylamino-pyridine (DMAP); and tertiary amine bases. Examples of amine bases include triethylamine (TEA), diisopropylethylamine (DIPEA), N-methyl-piperidine, 4-methylmorpholine, 2,6-lutidine, 1,2,2,6,6,-pentamethylpiperidine, N,N′-diethylaniline, diazabicyclononane (DBN), diaminocyclohexane, diethylamine, ethanolamine, DABCO, proton sponge, N,N,N′,N′-tetramethyl-1,8-naphthalenediamine, or azabicycloundecenes, such as 1,8-Diazabicyclo[5.4.0]undec-7-ene (DBU) or 1,5,7-Triazabicyclo[4.4.0]dec-5-ene (TBU). Solid-phase resin bases include resins based on tertiary amines, ammonium bases, or ion exchange resins. In another aspect, no base need be used to form Compound 1, such as in reactions in Scheme 1 and Scheme 2. Bases that are in insoluble in an organic solvent such as used in Scheme 1 or Scheme 2 may also be used.

In other embodiments, the conversion of Compounds 2 and 3, 10, or 18 to Compound 1 and/or 16 can be accomplished at a temperature above room temperature. In one aspect, the temperature can be up to about 200° C., about 100° C., about 70° C., or about 35° C. In other aspects the temperature can be from about 15° C. to about 35° C.; and then from about 15° C. to about 100° C. or about 70° C.

Compound 2 described herein may be prepared for example by the process comprising the steps of:
reacting Compound 4

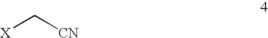

wherein X is Cl, Br, or I;
with Compound 5:

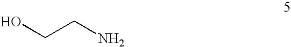

in the presence of a second base to yield Compound 6 or a salt thereof:

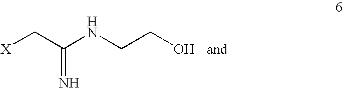

cyclizing Compound 6 or its salt in the presence of a third base to yield Compound 2 or a salt thereof.

The reaction may be carried out, for example, in an organic solvent, for example methanol.

An example of the third base is potassium t-butoxide.

The cyclization may be performed in, for example, t-butanol.

The salt of compound 6 may be, for example, the hydrochloride salt.

Alternatively, Compound 2 may be prepared by the process comprising the steps of:
reacting Compound 4

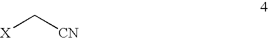

with Compound 5:

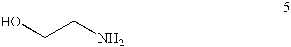

in the presence of a second base to yield Compound 6:

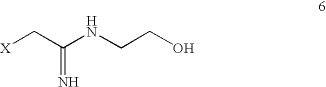

treating Compound 6 with hydrochloric acid to yield a hydrochloride salt, Compound 7:

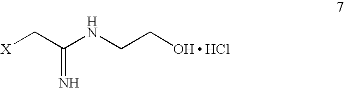

and cyclizing Compound 7 in the presence of a fourth base to yield Compound 2 or a salt thereof, wherein X is Cl, Br, or I.

At least one step in the process may be performed, for example, in an organic solvent.

Compound 3 as described herein may be prepared, for example, by a process comprising the steps of:
halogenating Compound 8:

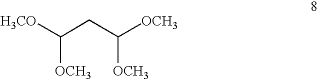

in the presence of a halogenating agent and a first acid to yield Compound 9:

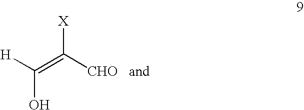

reacting Compound 9 with isopropanol in the presence of a catalytic amount of a second acid to yield Compound 3, wherein X is Cl, Br, or I.

The first acid may be, for example, hydrochloric acid.

The final step, to yield Compound 3, may for example further comprise refluxing methylcyclohexane or cyclohexane.

An example of a process to prepare Compound 2 or its acetate salt, 17, is shown in Scheme 3. In Scheme 3, suitable reaction conditions are shown, however, other reaction conditions may be used within the scope of the invention. For example, shorter or longer reaction times may be employed; generally the longer the reaction time, the more complete the reaction; and other bases and organic solvents may be employed.

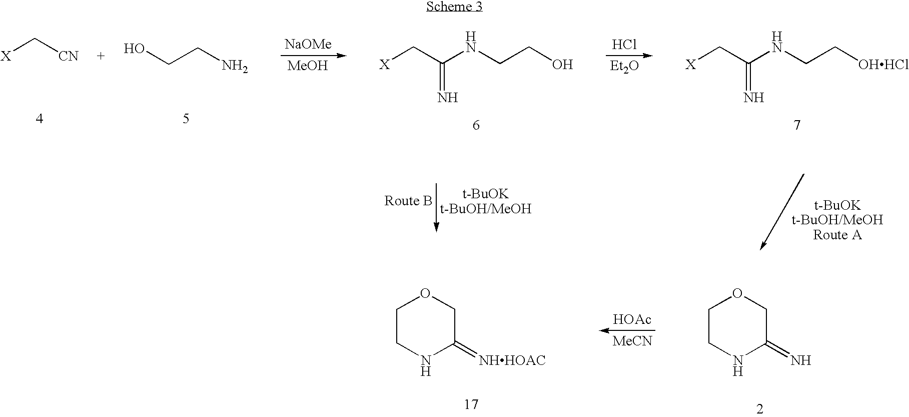

In one embodiment, shown in Scheme 3, Compound 6 can be prepared by reacting an activated acetonitrile where X is Cl, Br, or I, such as chloroacetonitrile (4, where X is Cl), with an ethanolamine (5) under basic conditions in an organic solvent to yield an activated acetamidine, such as chloroacetamidine (6, where X is Cl). The basic conditions can include a base, such as catalytic sodium methoxide in an anhydrous organic solvent such as anhydrous methanol. The reaction temperature to form the activated acetamidine can be from about 15° C. to about 70° C., or from about 20° C. to about 35° C. The resultant acetamidine (6) can be converted to a salt (7) by treatment with an acid, such as HCl, in an organic solvent, such as anhydrous diethyl ether. The salt formation can be accomplished at a temperature of at or below room temperature, such as from about −10° C. to about 20° C., or from about 0° C. to about 15° C. Compound 7 can then be cyclized to Compound 2 under basic conditions, such as with sodium methoxide or potassium t-butoxide. For example, Compound 2 can be formed from the salt (7) as indicated in ROUTE A in Scheme 3. However, Compounds 6 and 7 are reactive, so care should be taken when they are handled, such as by storage in a refrigerator under an anhydrous atmosphere such as anhydrous nitrogen. Alternatively, Compound 6 can be cyclized directly to Compound 2 under basic conditions that include a base such as potassium t-butoxide, in an anhydrous organic solvent such as t-butanol or a mixture of t-butanol and methanol, as indicated in ROUTE B in Scheme 3. ROUTE B avoids the isolation of Compound 7. Alternatively, Compounds 4 and 5 can be converted to Compound 2 in one pot, without isolating Compound 6 or Compound 7. The conversion of Compounds 4 and 5 to Compound 6 may proceed through Intermediate 1:

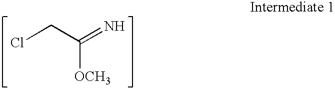

In one embodiment, a catalytic amount of base can be used in forming the acetamidine (6). The catalytic amount can be from about 0.5 to about 0.001 equivalents of base. In a specific embodiment, about 0.05 equivalents of a base such as sodium methoxide is used.

In some embodiments, Compound 4 can be activated acetonitriles, such as iodoacetonitrile, bromoacetonitrile, and chloroacetonitrile.

In other embodiments, Compound 2 can be converted to a salt such as an acetate salt (17), by treating a mixture that includes Compound 2 with an acid, such as acetic acid. For example, a crude reaction mixture containing Compound 2 in acetonitrile, below room temperature, such as at about 10° C., can be treated with 1 equivalent of acetic acid or more and stirred at or below about room temperature for about an hour. The precipitated acetate salt 17 can be filtered off, washed with acetonitrile, and dried. Converting Compound 2 to a salt, such as Compound 17, enables the enhanced isolation of Compound 2 from its reaction mixture. The salt (17) can be used in Scheme 1 or Scheme 2, above, in place of Compound 2. Compound 2 or its salt can degrade, so care should be taken in handling, such as by storage in a refrigerator or freezer under an anhydrous atmosphere, such as anhydrous nitrogen, in the dark.

In one aspect, an organic solvent used in a reaction of Scheme 3 can be anhydrous. An organic solvent used in a reaction in Scheme 3 can be a polar, aprotic organic solvent such as acetonitrile (MeCN) or tetrahydrofuran (THF). In some aspects the solvent can be a protic organic solvent such as an alcohol such as methanol, ethanol, isopropanol, or t-butanol. In another aspect, the solvent can be a non-polar organic solvent, such as an ether such as diethyl ether or MBTE [what does acronym stand for], or toluene, ethylacetate, or any combination thereof. Also, methylene chloride (DCM) [dichloromethane?] can be used in Scheme 3.

In one embodiment, Compound 6 or its salt can be prepared or isolated at a temperature of from about 10° C. to about 100° C., about 20° C. to about 35° C., or the initial temperature can be about 20° C. to about 25° C. and then rise to about 30° C. to about 35° C., or the temperature can be about 70° C. In another embodiment, the reaction to prepare Compound 7 can be accomplished at a temperature from about −10° C. to about 20° C., about 0° C. to about 15° C., about 0° C. to about 15° C. and then about room temperature, about 20° C. to about 35° C., or from about 20° C. to about 25° C.

In another embodiment, Compound 2 can be prepared at a temperature of from about 10° C. to about 100° C. In yet other embodiment, the temperature of the reaction can be from about 40° C. to about 70° C., or from about 50° C. to about 60° C. In one aspect, the isolation of Compound 2 from the reaction mixture can be accomplished at a temperature from about 0° C. to about 50° C., such as from about 10° C. to about 15° C.

In other embodiments, the conditions for the cyclization to yield Compound 2 can be more basic, i.e. at a higher pH, than the conditions for acetamidine (6) formation. The pKa of the base used in the cyclization step can be higher than the pKa of the base used in forming the acetamidine. For example, the pKa of the base used in the cyclization step can be from about 1 to about 5, or up to about 10 or 15 units higher than the pKa of the base used in forming the acetamidine. In another embodiment, the base used to form the acetamidine is nucleophilic. In yet another embodiment, the base used in the cyclization step to form Compound 2 is non-nucleophilic, or less nucleophilic than the base used in forming the acetamidine (6).

In some embodiments, an acid used in a reaction of Scheme 3 can be an organic or inorganic acid. Inorganic acids include hydrochloric (HCl), hydrobromic (HBr), sulfuric (H₂SO₄), perchloric (HClO₄), phosphoric (H₃PO₄), or solid-phase, such as resin-based, acids. Organic acids include carboxylic acids, such as acetic acid, benzoic acid, or oxalic acid. Other acids useful in the invention include sulfonic acids such as tosic acid or benzenesulfonic acid. In one embodiment, a salt of a compound of the processes of the invention is formed using an organic or inorganic acid. Acids that lead to pharmaceutically acceptable salts of compounds of the invention may also be used.

In one embodiment, a compound disclosed herein can be in a salt form, such as a hydrochloride or acetate salt. In other embodiments, the salt 7 or 17 is an inorganic salt. In yet other embodiments, the salt 7 or 17 is an organic salt, such as an acetate salt. In still other embodiments, the salt 7 or 17 is a pharmaceutically acceptable salt.

In some embodiments, inorganic salts include a hydrochloride, hydrobromide, hydrofluoride, hydroiodide, or sulfate salt.

A representative example of a process to prepare an activated compound such as Compound 3 is shown in Scheme 4. In Scheme 4, suitable reaction conditions are shown, however other reaction conditions may be used within the scope of the invention. For example shorter or longer reaction times may be employed; generally the longer the reaction time, the more complete the reaction, and other bases and organic solvents may be used.

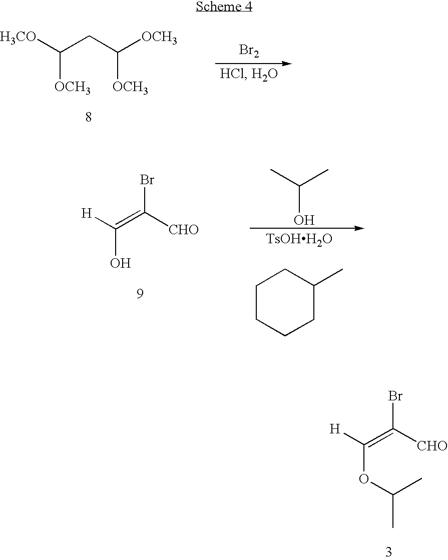

In an embodiment, such as is shown in Scheme 4, a dialdehyde, cyclic acetal, or acetal such as malonaldehyde-bis-dimethylacetal (8) can be activated by halogenation with a halogenating agent, such as a molecular halogen such as bromine, iodine, or chlorine, or NBS, NIS, or NCS, under acidic conditions, such as in catalytic hydrochloric acid in water, to obtain an intermediate halogenated aldehyde, such as chloro-, iodo-, or bromomalonaldehyde (9). The resultant halogenated aldehyde can be converted to an activated alkoxyacrylaldehyde, such as chloro-, iodo-, or bromo-alkoxyacrylaldehyde (3), and such as a C₁₋₆alkyl ether such as an isopropyl (Compound 3), methyl, ethyl, n-propyl, or butyl ether, by treatment with an alcohol and catalytic acid. As shown in Scheme 4, one embodiment of conditions for the exchange employ combining Compound 9 with isopropanol and catalytic para-toluenesulfonic (tosic) acid (TsOH) hydrate in an organic solvent, such as methylcyclohexane, cyclohexane, dichloromethane, or toluene, and refluxing the reaction using a Dean-Stark trap to aid the removal of water. The reaction mixture containing Compound 3 can be concentrated to increase the yield of Compound 3 that is isolated. While Compound 2 may be used in process of Scheme 1, the use of a protected version of Compound 2, such as Compound 3, reduces unwanted by-products, such as polymers, from the reaction of Scheme 1.

An organic solvent as used in Scheme 4 can be aprotic and high boiling, i.e. have a boiling point above or near that of water. Such organic solvents useful in a reaction yielding Compound 3 include methylcyclohexane, cyclohexane, toluene, nitrated solvents such as nitrobenzene or nitromethane, halogenated aryls such as monochlorobenzene or dichlorobenzene, xylenes, including ortho-xylene, meta-xylene, para-xylene, or ethylene glycol, and the solvent can be anhydrous. The conversion of Compound 9 to Compound 3 should be accomplished under anhydrous conditions, such as by removing water from the indicated tosic acid hydrate, such as by drying under vacuum or azeotroping water away from the hydrate using an appropriate solvent, such as toluene, methylcyclohexane, cyclohexane, or a solvent listed above.

The acidic conditions in a reaction of Scheme 4 include an acid. The conditions for the first reaction also include an aqueous solvent, such as water, or an organic solvent. The conversion of a compound such as 8 to a compound such as 9 can employ an inorganic acid. Inorganic acids include hydrochloric (HCl), hydrobromic (HBr), sulfuric (H₂SO₄), perchloric (HClO₄), sulfonic such as tosic or benzenesulfonic, or solid-phase, such as resin-based, acids. The conversion of a compound such as 8 to a compound such as 9 can employ an excess or a stoichiometric amount of an acid, such as hydrochloric, hydrobromic, hydrofluoric, hydroiodic, or sulfuric acid. The conversion of a compound such as 9 to a compound such as 3 can employ an inorganic or organic acid and an organic solvent, such as an aprotic organic solvent with a boiling point above or near that of water, such as methylcyclohexane, cyclohexane, or totuene, or another high-boiling solvent listed above. Organic acids include carboxylic acids, such as acetic acid, benzoic acid, or oxalic acid. The conversion of a compound such as 9 so a compound such as 3 can employ a catalytic amount of acid, such as a sulfonic acid such as tosic or benzenesulfonic acid. A reaction of Scheme 4 can employ a catalytic amount of acid, a stoichiometric amount of acid, or an excess of acid, using methods known to one of skill in the art. Pharmaceutically acceptable acids, such as acids that are capable of yielding a salt as listed above, may also be used in one or more reactions of Scheme 4.

In one embodiment, the acid can be present in a catalytic amount, such as from 0.001 to 0.2 equivalents of acid, from about 0.01 to about 0.15 equivalents, about 0.01 equivalents, or from about 0.1 to about 0.15 equivalents. In other embodiments, 0.001 to 0.15 equivalents of catalytic acid relative to Compound 9 can be used in a reaction of Scheme 4. In yet other embodiments, about 0.01 equivalents of catalytic acid can be used.

In one aspect, Compound 9 can be isolated. The precipitate can be washed with water at or below about room temperature, filtered, and the solid dried, such as with a forced-air dryer. In another aspect, Compound 3 can be isolated, such as by distillation of the reaction solvent away from the product.

In an embodiment, the reaction to prepare Compound 3 and its isolation are accomplished at a temperature of less than about 50° C., or at from about 0° C. to about 50° C. Maintaining the temperature below about 50° C. can reduce the decomposition of Compound 9 or Compound 3 during its formation or isolation. In some embodiments, the temperature can be from about 0° C. to about 45° C., about 5° C. to about 30° C., or about 5° C. to about 25° C. In one aspect, the temperature of isolation of Compound 7 can be less than about 50° C., about 45° C., or about room temperature.

Other embodiments includes forming an activated compound such as Compound 3, 9, 10, or 18 in situ, without the isolation or purification of intermediates before the next synthetic step.

In some embodiments, a process of the invention, Scheme 1, 2, 3, 4, or 5, or any step therein, does not employ morpholine-3-one; morpholine-3-thione; 5-methylthio-3,6-dihydro-2H-[1,4]oxazine; 3-iminomorpholine hydrochloride; Lawesson's reagent; methyl iodide; cyclohexane; an alkali alkoxide such as sodium methoxide or potassium t-butoxide; an organic tertiary amine base such as triethylamine or diisopropylethylamine; or silica gel chromatography.

Compound 1 can be useful in the synthesis of penem β-lactamase inhibitors, such as a compound of formula 12, its E isomer, a mixture thereof, or a pharmaceutically acceptable salt or hydrate thereof, such as a sodium or potassium salt:

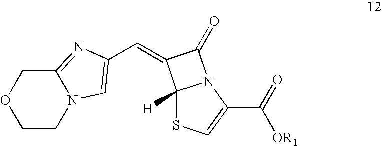

wherein
R₁ is H, an in vivo hydrolyzable ester selected from the group C₁₋₆alkyl, C₅₋₆cycloalkyl, —CHR₂OC(O)C₁₋₆alkyl, or a salt selected from the group of Na, K, and Ca; and R₂ is hydrogen, C₁₋₆alkyl, C₃₋₆cycloalkyl, optionally substituted C₃₋₁₄ aryl, or optionally substituted heteroaryl.

In accordance with the above, Compound 1 may be employed in a process comprising the steps of:
condensing Compound 1:

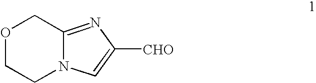

with a 6-bromo-penem derivative of formula 13:

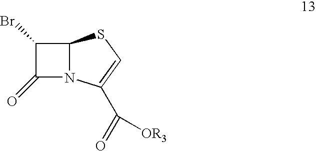

in the presence of a acid and a fifth-base,
wherein R₃ is para-nitrobenzyl, benzyl, para-methoxy benzyl, benzhydrol, or trityl, to form an intermediate aldol product of formula 14:

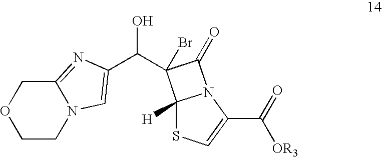

wherein R₃ is as defined above;
reacting the intermediate aldol product of formula 14 with an acid chloride of the formula R₄Cl, an anhydride of the formula (R₄)₂O, or C(X₁)₄ and triphenylphosphine,
wherein R₄ is C₁₋₆alkyl-SO₂—, C₃₋₁₄aryl-SO₂—, C₁₋₆alkyl-C(O)—, or C₃₋₁₄aryl-C(O)—; and X₁ is Br, I, or Cl, to form an intermediate of formula 15:

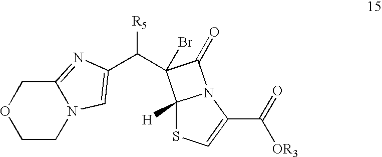

wherein R₃ is as defined above and R₅ is —OR₄ or X₁; and
converting the intermediate of formula 15 to the compound of formula 12, or a pharmaceutically acceptable salt or hydrate thereof, by a reductive elimination process.

The fifth base may be, for example, an organic base, for example triethylamine, DMAP or diisopropyl ethyl amine.

Examples of R₅ include acetate, triflate, or tosylate. An example of R₃ is para-nitrobenzyl.

The reductive elimination process may be carried out, for example, using activated zinc and a phosphate buffer at a pH of about 6.5 to 8.0 or hydrogenation in the presence of a catalyst.

The process may further comprise converting compound of formula 12 to a pharmaceutically acceptable salt, or an in vivo hydrolyzable ester selected from a C₁₋₆alkyl ester, a C₅₋₆cycloalkyl ester, and a —CHR₂OCOC₁₋₆alkyl ester, wherein R₂ is as defined above. For example, Compounds of the general formula 12 can be prepared in a mild and facile way as described below, for example as shown in Scheme 5. In Scheme 5, suitable reaction conditions are shown, however other reaction conditions may be used within the scope of the invention. For example, shorter or longer reaction times may be employed; generally the longer the reaction time, the more complete the reaction, and other bases and solvents may be used.

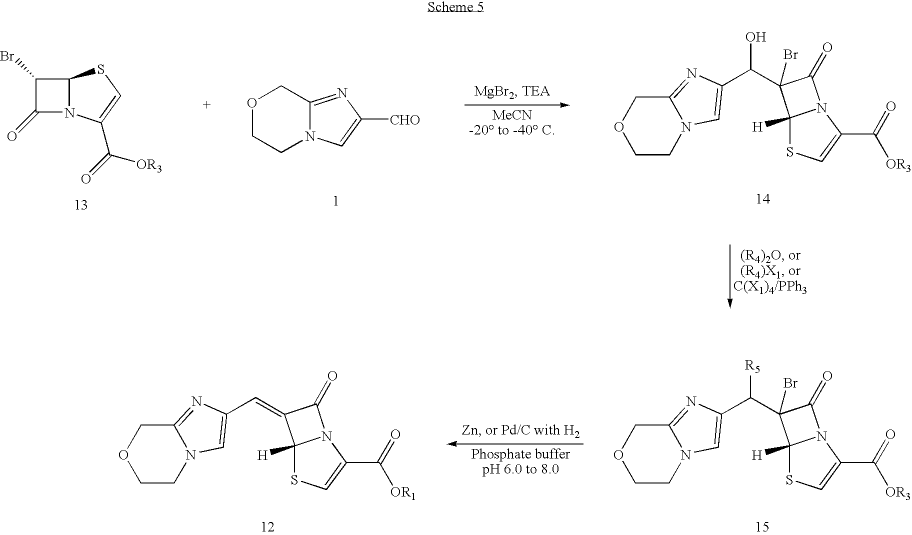

where R₁ is as defined above; R₃ is a protecting group that is hydrolyzable, by reaction with zinc or by hydrogenation in the presence of a catalyst such as with palladium on carbon, such as para-nitrobenzyl, benzyl, para-methoxy benzyl, benzhydrol, trityl, allyl, or an optionally substituted aryl or heteroaryl; alternatively R₃ may be a C₁₋₆ alkyl or a C₅₋₆ cycloalkyl; R₄ is C₁₋₆alkyl-SO₂—, aryl-SO₂—, C₁₋₆alkyl-C(O)—, or C₆₋₁₄aryl-C(O)—; X₁ is Br, I, or Cl; and R₅ is —OR₄ or X₁. R₃ may be hydrolyzed using conditions including zinc, or conditions including palladium and hydrogen, such as in the presence of 5% or 10% palladium on charcoal and under hydrogen at about 40 pounds per square inch (psi). The hydrolysis of R₃ may be carried out in an aqueous medium, such as water or phosphate buffer.

In one embodiment, as shown in Scheme 5, compounds of the general formula 12 can be prepared by condensing an aldehyde such as Compound 1 with a 6-bromo-penem derivative of formula 13 in the presence of a Lewis acid, preferably anhydrous magnesium halide and more preferably anhydrous MgBr₂ or MgBr₂.etherate and a mild base such as triethylamine, 4-dimethylamino-pyridine DMAP, or diisopropyl ethyl amine, at low temperature, preferably at about −20° C. to −40° C. The 6-bromo-penem derivative of formula 13 can be prepared as disclosed in U.S. Pat. No. 7,112,582 and U.S. Patent Publication Nos. 2004/0132708, 2004/0053913, and 2006/0217361, each of which is incorporated by reference in its entirety. The intermediate aldol product 14 can be functionalized with acid chlorides or anhydrides preferably to an acetate, a triflate or a tosylate of formula 15. Alternatively, if formula 14 is isolated, it can be converted to a halogen derivative by reacting 14 with tetrahalomethane and triphenylphosphine at room temperature in a suitable organic solvent preferably methylene chloride (DCM). Compound 15 can be smoothly converted to the desired product by a reductive elimination process using a metal such as activated zinc and a buffer such as phosphate buffer at mild temperatures, preferably about 20° C. to about 35° C., at a pH of about 6.5 to 8.0 or by hydrogenating over a catalyst, preferably palladium on charcoal. It should be noted that the reductive elimination step can be conducted such that deprotection of the carboxyl group also occurs. If the protecting group on the carboxylate oxygen is a para-nitrobenzyl substituent, then the reductive elimination and deprotection can be achieved by a single step. However, if the protecting group is other than para-nitrobenzyl, a two-step procedure can be followed depending up on the nature of the protecting group. The other protecting groups can include para-methoxy benzyl, benzhydrol, trityl, allyl or alkyl. The product can be isolated as a free acid or as an alkali metal salt. The above-mentioned two-step procedure can also be accomplished in one step by carrying out the entire process without isolating the intermediate 15. This is a relatively simple procedure and extremely efficient in terms of yield and economic feasibility, and can be used to make a wide variety of compounds. This procedure is also amenable to large scale synthesis and applicable to a variety of aldehydes, including Compound 1.

The above mentioned aldol condensation reaction is very versatile and it can be applied to any bromopenem derivative, where the carboxy group is protected other than para-nitrobenzyl moiety. Examples of other protecting groups include benzyl, para-methoxy benzyl derivative, benzyhydrol, trityl, alkyl and allyl derivatives. However, when the protecting group is other than para-nitrobenzyl group, a separate deprotection step needs to be carried out after the reductive elimination procedure. Protection of the carboxyl group with a para-nitrobenzyl group reduces the number of steps in the present process for preparing the final compound of formula 12. The chemistry involved in the deprotection step is well known to people who are skilled in the art.

In another embodiment, a 6-bromo-penem derivative of formula 13 can be prepared as disclosed in U.S. Pat. No. 7,112,582 or U.S. Patent Publication Nos. 2004/0132708, 2004/0053913, or 2006/0217361, each of which is incorporated by reference in its entirety.

In one embodiment, R₁ is H.

In other embodiments, R₁ is a salt, such as an organic or inorganic cation. In some embodiments, inorganic cations include monovalent metal ions, such as sodium, potassium, or lithium, or divalent metal ions where one metal ion is present with two penem derivatives of formula 12, such as calcium or magnesium. In other embodiments, organic cations can include ammonium ions, and the like.

In yet other embodiments, R₁ is a pharmaceutically acceptable salt.

In some embodiments, R₁ is an in vivo hydrolyzable ester.

In some embodiments, R₁ is para-nitrobenzyl.

In other embodiments, R₁ is R₃ or C₁₋₆ branched or straight-chain alkyl.

In still other embodiments, R₃ is para-nitrobenzyl or C₁₋₆ branched or straight-chain alkyl.

In other embodiments, R₄ is C₁-C₆ alkyl-SO₂, aryl-SO₂, alkyl-CO, or aryl-CO.

In other embodiments, R₅ is —OR₄ or X₁.

In still other embodiments, X₁ is Br, I, or Cl.

In an embodiment, each basic condition of a step can independently include an organic or inorganic base.

In other embodiments, a basic condition of a reaction disclosed herein can independently include an organic base. In other embodiments, the organic base can be a tertiary amine base such as pyridine or pyridine derivatives including 4-dimethylamino-pyridine (DMAP), triethylamine (TEA), diisopropylethylamine (DIPEA), N—C₁-C₆alkyl-piperidine such as N-methyl-piperidine or N-ethyl-piperidine, N—C₁-C₆ alkyl-morpholine such as N-methyl-morpholine or N-ethyl-morpholine, DABCO, N,N,N′,N′-tetramethyl-1,8-naphthalenediamine, or azabicycloundecenes, such as 1,8-Diazabicyclo[5.4.0]undec-7-ene (DBU) or 1,5,7-Triazabicyclo[4.4.0]dec-5-ene (TBU). Solid or liquid bases can be used, including solid-phase resin bases such as those containing tertiary or quaternary amines.

In other embodiments, a basic condition can independently include an inorganic base. In some embodiments, inorganic bases include alkali hydroxides, alkali oxides, or alkali carbonates, such as lithium, sodium, potassium, cesium, calcium, or magnesium salts of hydroxides, oxides, or carbonates. In yet other embodiments, alkali alkoxides include alkali metal salts of methoxide, ethoxide, propoxide, butoxide, or t-butoxide, with the proviso that Scheme 1 cannot employ an alkali alkoxide. Alkali metal salts include sodium, lithium, potassium, barium, cesium, or calcium salts.

In some embodiments, solid-phase resin bases including resins based on tertiary or quaternary amines, may also be used.

In yet other embodiments, where a base used in a reaction herein includes a base water-sensitive base such as sodium hydride or butyl lithium, a protic solvent such as water or an alcohol is not also present.

In some embodiments, all or a plurality of the steps of the processes of the invention can be performed in one pot. In yet other embodiments, the steps of Scheme 1 can be carried out in one pot, without the isolation of intermediates. In other embodiments, the steps of Scheme 2 can be carried out in one pot, without the isolation of intermediates. In some embodiments, the steps of Scheme 3 can be carried out in one pot, without the isolation of intermediates. In some embodiments, the steps of Scheme 4 can be carried out in one pot, without the isolation of intermediates. In some embodiments, the steps of Scheme 5 can be carried out in one pot, without the isolation of intermediates. In other embodiments, any or all intermediates of the process, except or including Compound 1, may be made in situ, without isolation of the intermediate before the next or a concurrent synthetic step is performed.

In some embodiments, where absolute stereochemistry of a compound or genus is shown, the processes of the invention include preparing the depicted enantiomer, the R or the S enantiomer, or a mixture thereof, including a racemic mixture.

In some embodiments, one or more steps of the processes of the invention can be carried out at about room temperature. In other embodiments, the temperature of the reaction can be above room temperature. In another embodiment, the temperature of the reaction can be below room temperature.

In one aspect, the term ‘anhydrous,’ when referring to reagents, organic solvents, reaction conditions, or atmospheres such as nitrogen (N₂), means substantially moisture-free, as will be apparent to one of skill in the art. Typically, anhydrous, when referring to organic solvents, means having about 10 to 30 ppm of water, or less, or about 0.005% water, or less.

In one embodiment, the term ‘low,’ when referring to temperature can mean below room temperature, and typically means below about 0° C., below about −20° C., below about −40° C., or at or below about −78° C. For example, a low temperature can be about −20° C. to about −40° C., or about −78° C.

The below experimental procedures are intended to illustrate some embodiments of the present invention and are not intended to limit the scope of the invention.

### EXPERIMENTAL PROCEDURES

### Example 1

### Preparation of 2-Chloro-N-(2-hydroxy-ethyl)-acetamidine hydrochloride salt (Compound 7, where X is Cl)

Chloroacetonitrile (4) (271.0 grams (g), 3.59 mol) was dissolved in anhydrous methanol (2600 milliliter (mL)) in a 12 liter (L), four-neck flask equipped with a mechanical stirrer, thermocouple, and condenser, nitrogen inlet, and condenser, with stirring under a nitrogen atmosphere. To the resultant clear solution at 20° C. to 25° C., 25 wt-% sodium methoxide in methanol (38.8 g, 0.179 mol) was added drop-wise over about 30 minutes while the temperature gradually rose to about 30° C. to 35° C. The mixture was stirred for 15 to 30 minutes and a solution of ethanolamine (5) (219.2 g, 3.59 mol) in anhydrous methanol (120 milliliter (mL)) was added to the flask slowly over about 45 minutes. The mixture was stirred over twelve hours at room temperature and monitored by GC for reaction completion. The mixture was then cooled to 0° C. to 5° C. and 2.0 Normal (N) hydrochloric acid (HCl) in anhydrous diethyl ether (1885 mL, 3.77 mol HCl) was added slowly over about 2 hours while the temperature was maintained between 0° C. and 15° C. The mixture was allowed to warm to room temperature and stirred for at least 1 hour. The product (7, where X is Cl) mixture was concentrated under reduced pressure to about 700-800 grams of purple oil and stored under nitrogen in a refrigerator. Care was taken in handling the product because it may be explosive. Mass spectrometry (M+H): 108.54 atomic mass units (amu)

### Example 2

### Preparation of Morpholin-3-ylideneamine, Compound 2

A solution of 2-chloro-N-(2-hydroxy-ethyl)-acetamidine hydrochloride salt (7, where X is Cl) (406 g, 310.0 g real, 1.79 mol) in about 96 grams of methanol and anhydrous tert-butanol (4200 mL) was added to a 12-L, four-neck flask equipped with a mechanical stirrer, thermocouple, nitrogen inlet, and condenser, and the mixture was stirred under a nitrogen atmosphere. To the resultant purple solution, 402.1 g (3.58 mol) of potassium tert-butoxide was added portion wise over 1 to 1.5 hours to the flask while the temperature rose to 50° C. to 60° C. The mixture was stirred for about 45 minutes and a nuclear magnetic resonance (NMR) spectrum was taken of a sample of the reaction to check for disappearance of the starting material. The batch was allowed to cool to 25° C. to 40° C. and 4000 mL of anhydrous acetonitrile were added over about 30 minutes while the mixture endothermed to about 10° C. to 15° C. The salts were filtered off on a 24 centimeter (cm) Buechner funnel and washed with 2-3 L of fresh acetonitrile. The combined filtrates were filtered and the salts collected were washed with 2-3 L anhydrous acetonitrile to minimize residual solids. The filtering and washing process was repeated once or twice more to reduce the content of residual solids. The final, combined filtrates were concentrated under reduced pressure to about 800 to 1000 grams and re-filtered on a 12 cm Buechner funnel. The salts collected were washed with acetonitrile. The combined filtrates were transferred to a 3 liter flask and concentrated to a brown oil-solid mixture to afford about 141 g crude product, in about a 79% crude yield. The concentrate was filtered again if solids appeared during the concentration. The product (2) was stored under nitrogen in the refrigerator. Mass spectrometry (M+H): 101.12 atomic mass units (amu)

### Example 3

### Integrated Step for the Preparation of Morpholin-3-ylideneamine (2)

Chloroacetonitrile (4) (226.5 g, 3.0 mol) was dissolved in anhydrous methanol (1000 mL) in a 12-L, four-neck flask equipped with a mechanical stirrer, thermocouple, nitrogen inlet, and condenser, and the mixture was stirred under a nitrogen atmosphere. To the resultant clear solution at 20° C. to 25° C., 25 wt % sodium methoxide in methanol (32.4 g, 0.15 mol) was added drop-wise over about 30 minutes to the flask while the temperature gradually rose to 30° C. to 35° C. The mixture was stirred for 15 to 30 minutes and a solution of ethanolamine (5) (183.2 g, 3.0 mol) dissolved in 120 mL anhydrous methanol was slowly added to the flask over about 45 minutes. The mixture was stirred overnight (more than 12 hours) at room temperature and monitored by GC for reaction completion. Potassium tert-butoxide was then added to the reaction portion wise over 45 minutes to 1 hour while the temperature rose to 50° C. to 60° C. The mixture was stirred for about 45 minutes and an NMR was taken of a sample of the reaction to check for disappearance of starting material. The batch was allowed to cool to 25° C. to 40° C. and about 2600 mL of acetonitrile was added over about 30 minutes while the temperature endothermed to about 10° C. to 15° C. The salts were filtered off using a 24 cm Buechner funnel and washed with about 2 liters of acetonitrile. The filtrate was re-filtered 2 or 3 more times until the salts were mostly removed and then concentrated to a dark mixture of about 720 grams of crude, dilute product in acetonitrile. The product was stored under nitrogen in the refrigerator.

### Example 4

### Preparation of Morpholin-3-ylideneamine Acetate Salt (17)

Methanol (400 mL) and 25% sodium methoxide (10.8 g, 0.05 mol) were added to a 2-L multiple necked flask under nitrogen. The mixture was cooled to 10° C. and chloroacetonitrile (75.5 g, 1.0 mol) was added to the stirred solution over 0.5 hours at 10° C. to 20° C. The resulting solution was held for an hour at 10° C. to 20° C. to give Intermediate 1.

Ethanolamine (61.1 g, 1.0 mol) was added over 15 minutes at 10° C. to 20° C. The solution was stirred over night (17 hours) at room temperature to give a purple solution. The reaction solution was cooled to 10° C. and 25% sodium methoxide (227 g, 1.05 mol) was added over 30 minutes at 10° C. to 20° C. The mixture was heated to 50° C. to 55° C. and held for an hour to give Compound 2. The mixture was then cooled to 20° C., filtered to remove salts, and the filter cake washed with 100 mL of methanol.

The methanol was removed in vacuo at 20° C. to 50° C. and 40 to 50 torr to give a brown oily residue (129.7 g) containing Compound 2. To remove tars, the oily residue was added with stirring to a 2-L flask containing acetonitrile (1.3 L) and magnesium sulfate (54 g). The mixture was stirred at room temperature for 0.5 hours and filtered to remove tars and magnesium sulfate. The filter cake was washed with 200 mL of acetonitrile. The combined filtrate and wash were cooled to 10° C., acetic acid (27.0 g, 0.45 mol) was added, and the mixture was stirred for an hour at 10° C. to 20° C. The mixture was filtered at 10° C. and the filter cake was washed with acetonitrile (200 mL). The wet cake was dried in vacuo at 40° C. to 50° C. to give a white to off-white solid (47.6 g, 29.8% based on chloroacetonitrile) of the acetate salt, Compound 17. HPLC purity 98.9%. The product was stored in a freezer (−10° C. to −25° C.), in the dark.

### Example 5

### Preparation of Morpholin-3-ylideneamine (2) from the acetate salt (17)

Compound 17 (48.0 g, 0.3 mol) and methanol (200 mL) were added to a 500 mL flask. A 25% solution of sodium methoxide (68.4 g, 0.3 mol) was added over 5 minutes at room temperature and the mixture was stirred for 0.5 hours. The methanol was removed in vacuo at 20° C. to 50° C. and 40 to 50 torr. Methylene chloride (250 mL) was added to the resulting residue. The mixture was then filtered to remove sodium acetate and the filter cake was washed with methylene chloride (100 mL). The methylene chloride was removed in vacuo at 20° C. to 50° C. and 40 to 50 torr to give 27.5 g (91.6%) of white to off-white solids of the free base, Compound 2.

### Example 6

### Preparation of Bromomalonaldehyde (9)

De-ionized water (2.080 L) was added to a 12-L, four-neck flask under a nitrogen atmosphere and equipped with a thermocouple. Concentrated HCl (88 mL) was added to the water. Malonaldehyde bis-dimethylacetal (2006 mL, 2.0 kilogram (kg), 12.18 moles) was added dropwise over a period of 45 to 60 minutes while the temperature was maintained between 5° C. and 25° C. Bromine (1.912 g, 619 mL; 12 moles; 1 equivalent.) was added dropwise over 1 hour, while the temperature was maintained between 5° C. and 20° C. The reaction was monitored by HPLC for completion. After completion of the reaction (2 to 4 hours), the reaction mixture was concentrated on a rotary evaporator at 45° C. and 100 torr. The resultant mixture was stirred for 1 hour at room temperature, filtered, and washed with cold water (2×1.0 liter). The isolated solids were dried in a forced-air dryer for 2 days. The weight of the product (9) obtained was 1.008 kg, with a 430 g second crop from the filtrate, giving a combined 76% yield. The second crop of crystals was obtained by concentrating the mother liquor to half its original volume and filtering off the resulting crystals. Mass spectrometry (M+H): 150.97 and 151.96 amu.

### Example 7

### Preparation of 2-Bromo-3-isopropoxy-propenal from bromomalonaldehyde (3)

Bromomalonaldehyde (9) (203 g, 1.34 mol), 2-propanol (257 mL, 3.34 mol), methylcyclohexane (1360 mL), and para-toluenesulfonic acid monohydrate (2.55 g, 0.01 equiv., 0.0134 mol) were added to a 5-L, four-neck flask equipped with a mechanical stirrer, thermocouple, a Dean-Stark trap with condenser, and a nitrogen inlet, and the mixture was stirred under nitrogen. The resultant orange-tan slurry was gradually heated to a gentle reflux. Distillation started at a pot temperature of about 77° C., and the temperature rose to about 87° C. over about 1 to 2 hours, while the vapor temperature gradually rose to about 78° C. When virtually no more water was observed collecting in the Dean-Stark trap, the azeotropic distillation was continued for about 1 hour to ensure reaction completion. The reaction mixture was then cooled and concentrated under reduced pressure to afford 243 g dark orange/brown oil, with about a 93% crude yield of product. Although two layers formed on cooling, solvent distillation was required to achieve high yield. NMR (in CDCl₃) showed mostly 1 isomer, but the product equilibrated on standing. The product was stored in the freezer under nitrogen. The product had an HPLC retention time of 7.82 minutes at a 220 nanometer (nm) UV wavelength using a C18 column with a mobile phase gradient from 95% 10 mM ammonium carbonate and 5% acetonitrile to 100% acetonitrile over 9 minutes at a flow rate of 1.0 mL/minute. This procedure has also been scaled up to 787 g in 12-liter glassware. Alternatively, cyclohexane has been used instead of the less-flammable methylcyclohexane. Mass spectrometry (M+H): 192.98 and 194.98 amu.

### Example 8

### Preparation of 5,6-Dihydro-8H-imidazo[2,1-c][1,4]oxazine-2-carbaldehyde (1)

Crude morpholin-3-ylideneamine (2) (159.0 g, estimated 60% to 75% purity, 1.59 mol) and acetonitrile (1336 mL) were added to a 5-L, four-neck flask equipped with mechanical stirrer, thermocouple, condenser and nitrogen inlet, and the mixture was stirred under nitrogen. 2-bromo-3-isopropoxy-propenal (3) (230 g, 1.19 mol) was dissolved in 690 mL acetonitrile, transferred to a 1-L dropping funnel, and slowly added to the flask over 1 hour to 1.5 hours while the temperature gradually rose to 30° C. to 35° C. The dark mixture was stirred and an HPLC of a sample was taken after 15 to 30 minutes to confirm intermediate formation. After stirring for about 1 hour, solid potassium carbonate (325 mesh) (178.8 g, 1.27 mol, 1.07 equiv.) was added, and the reaction was heated to about 70° C. An HPLC of a sample was taken after 15 to 30 minutes to confirm reaction completion. The stirring mixture was then allowed to cool to 20-30° C. The slurry of solid potassium carbonate (K₂CO₃) was filtered at room temperature and the solids collected washed with 400 mL acetonitrile. The mother liquors (weighing about 2 kg) were concentrated under reduced pressure (45° C. to 48° C.) to about 335 g of a dark viscous liquid. The concentrate was then partitioned between methylene chloride (DCM) (700 mL) and water (350 mL). The aqueous layer was extracted three times with 200 mL DCM (3×200 mL). The combined organic layers were filtered through silica gel (70 g) and the silica gel was washed with 400 mL DCM. The combined filtrates were concentrated until crystallization began. Then t-butyl methyl ether (TBME) was added and the TBME mixture was evaporated, yielding a final weight of about 312 g of slurry of Compound 1. This process was repeated until minimal methylene chloride remained in the orange slurry, as judged by no visible increase in crystallization or no visible decrease in the viscosity of the residual oil, which contained DCM and the regioisomer 16. The amount of methylene chloride may also be determined by NMR, for example. The slurry was filtered, washed with TBME, and dried at room temperature to afford about 60 g of yellow to orange colored product, yielding about 25% of 5,6-dihydro-8H-imidazo[2,1-c][1,4]oxazine-2-carbaldehyde (Compound 1).

Compound 1: Mass spectrometry (M+H): 130.21 amu. ¹H NMR (CDCl₃) δ 4.08-4.15 (m, 4H), 4.88 (s, 2H), 7.58 (s, 1H), 9.85 (s, 1H). The unwanted regioisomer (16): ¹H NMR (CDCl₃) δ4.06 (t, 2H, J=5.2 Hz), 4.40 (t, 2H, J=5.2 Hz), 4.90 (s, 2H), 7.75 (s, 1H), 9.72 (s, 1H).

### Example 9

### Alternative Preparation of 5,6-Dihydro-8H-imidazo[2,1-c][1,4]oxazine-2-carbaldehyde (1)

To a stirred solution of morpholin-3-ylideneamine hydrochloride salt (1.3 g, 10 millimolar (mmol)) in DME (50 mL) at room temperature was added an excess of anhydrous K₂CO₃ and the mixture stirred for 10 minutes. Then ethyl-bromopyruvate (10) (2.94 g, 15 mmol) was added and the mixture stirred at room temperature for 4 hours, then refluxed for 16 hours. Examination of TLC by UV visualization using 1:1:0.05 ethylacetate:hexane:methanol showed only one product. The reaction mixture was concentrated and extracted with chloroform. The chloroform extract was dried over anhydrous Na₂SO₄, filtered, and concentrated. The crude product was purified by SiO₂ column chromatography using 1:1:0.05 ethylacetate:hexane:methanol as eluent, yielding 980 mg of a pale yellow liquid (50% yield).

The intermediate ester (11) (600 mg, 3.06 mmol) was dissolved in anhydrous THF (50 mL) and cooled to −78° C. To the stirred reaction mixture, DIBAL (1 molar (M) solution, 3.5 mL) was slowly added. The reaction mixture was stirred for 2 hours while the temperature was slowly elevated to −40° C. and then stirred for 1 hour at 40° C. The reaction mixture was then quenched with a saturated NH₄Cl solution and extracted with chloroform. The chloroform extract was washed once with a saturated brine solution. The organic layer was dried over anhydrous Na₂SO₄ and filtered. It was concentrated and purified by SiO₂ column chromatography, eluting with ethylaceate:hexane (4:1), yielding 250 mg (41%) of the product Compound 1 having the same ¹H-NMR spectrum as above.

### Example 10

### Second Alternative Preparation of 5,6-Dihydro-8H-imidazo[2,1-c][1,4]oxazine-2-carbaldehyde(1)

A 0.05 g sample of morpholin-3-ylideneamine (2) (0.5 mmol, purity 70-80% by NMR) was stirred with 0.03 g of bromoaldehyde (0.15 mmol) at 30° C. for 30 to 60 minutes in 1.2 mL of an organic solvent. 1 mmol of base was added and the reaction heated to 70° C. for 30 minutes. The reaction was sampled and analyzed by HPLC using a C18 column, with a mobile phase gradient from 95% 10 mM ammonium carbonate and 5% acetonitrile to 100% acetonitrile over 9 minutes at a flow rate of 1.0 mL/min, and using UV detection at 220 nm and 264 nm. The product appeared as two broad peaks at 264 nm and 3.45 and 4.20 minutes, and the regioisomer appeared as a single peak at 264 nm and 5.75 minutes. Alternatively, the same weights of starting materials were used with 85 to 95% pure morpholin-3-ylideneamine (2) and 0.4 mmol of base. In all of the reaction mixtures, excess bromoaldehyde remained after heating. The organic solvents screened included acetone, N,N-DMAc, THF, ethyl acetate, ethyleneglycol diethyl ether, 1,2-dimethoxyethane, 1,2-dichloroethane, NMP, DMF, acetonitrile, DMSO, toluene, sulfolane, and ethanol, all of which yielded detectable amounts of product (1) and its regioisomer (16) in ratios ranging from about 0.4 to about 60. The bases screened with these solvents included lithium carbonate, cesium carbonate, 4-methylmorpholine, triethylamine, 2,6-lutidine, 2,2,6,6-tetramethyl-piperidine, diaminocyclohexane, N,N′-diethylaniline, DBN, pyridine, diethylamine, and ethanolamine; these bases had a small effect on the ratio of product (1) to its regioisomer (16). An absence of base also yielded product (1).

### Example 11

### Preparation of (5R), (6Z)-6-(5,6-Dihydro-8H-imidazo[2,1-c][1,4]oxazin-2-ylmethylene)-7-oxo-4-thia-1-azabicyclo[3.2.0]hept-2-ene-2-carboxylic acid, sodium salt (12)

An anhydrous acetonitrile (66 mL) solution of 5,6-dihydro-8H-imidazo[2,1-c][1,4]oxazine-2-carbaldehyde (1) (1.2 g) was added to an anhydrous acetonitrile (66 mL) solution of MgBr₂ (3.6 g) under a nitrogen atmosphere at room temperature and the mixture was stirred for 10 minutes. An anhydrous THF (132 mL) solution of para-nitrobenzyl (5R,6S)-6-bromopenem-3-carboxylate (3.4 g) was added and the mixture was cooled to −20° C., then triethylamine (2.8 mL) was added in one portion. The reaction vessel was covered with foil to exclude light. The reaction mixture was stirred for 4 hours at −20° C. and treated with 4-dimethylamino-pyridine (100 mg) and acetic anhydride (1.5 mL) in one portion. The reaction mixture was warmed to 0° C. and stirred for 18 hours at 0° C. A 10% Citric acid aqueous solution (1-L) was added to the reaction mixture and the aqueous layer was extracted with ethyl acetate (3×500 mL). The combined organic layer was washed with water, saturated sodium bicarbonate and brine, dried over MgSO₄ and filtered. The filtrate was concentrated under reduced pressure and crude (5R)-6-[acetoxy-(5,6-dihydro-8H-imidazo[2,1-c][1,4]oxazin-2-yl)methyl]-6-bromo-7-oxo-4-thia-1-azabicyclo[3.2.0]hept-2-ene-2-carboxylic acid para-nitrobenzyl ester was obtained as brown amorphous solid.

Freshly activated Zn dust (14 g) was added rapidly with 0.5 mol/L phosphate buffer (pH 6.5, 72 mL) to the THF (72 mL) solution of (5R)-6-[acetoxy-(5,6-dihydro-8H-imidazo[2,1-c][1,4]oxazin-2-yl)methyl]-6-bromo-7-oxo-4-thia-1-azabicyclo[3.2.0]hept-2-ene-2-carboxylic acid para-nitrobenzyl ester. The reaction vessel was covered with foil to exclude light. The reaction mixture was vigorously stirred for 2.5 hours at room temperature. The reaction solution was filtered through a pad of Celite and the pad was washed with water (170 mL) and n-butanol (170 mL). The aqueous layer was separated and then the organic layer was extracted with 0.5 mol/L phosphate buffer (pH 6.5, 2×50 mL). The combined aqueous layer was concentrated to 90 g and 1 mol/L NaOH was added to adjust pH to 7.5. The concentrate was applied to Diaion HP-21 resin (120 mL, Mitsubishi Kasei Co. Ltd.) for column chromatography. After adsorbing, the concentrate was eluted from the column with water followed by a 5% acetonitrile aqueous solution. The combined active fractions were concentrated under high vacuum at 35° C. and lyophilized to give the title compound as a yellow amorphous solid (756 mg, 29.1%). Mp: 130° C. (dec); ¹H NMR (DMSO-d₆) δ 3.98-4.01 (m, 2H), 4.04-4.07 (m, 2H), 4.74 (AB, 2H, J=15.3, 22.9 Hz), 6.40 (d, 1H, J=0.8 Hz), 6.55 (s, 1H), 6.95 (d, 1H, J=0.6 Hz), 7.54 (s, 1H); IR (KBr) 3412, 1741, 1672, 1592, 1549 cm⁻¹; λₘₐₓ(H₂O) 304 nm.

What is claimed is:

1. A process for preparing Compound 1: 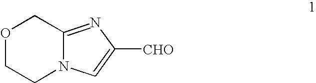
the process comprising the steps of:
reacting Compound 2 or a salt thereof: 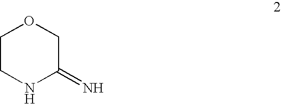
with Compound 3: 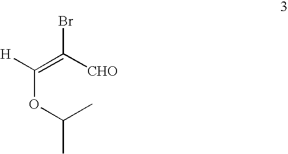
in the presence of a first base, selected from an alkali carbonate and an amine base, to yield Compound 1.

2. A process as claimed in claim 1, further in the presence of an organic solvent.

3. A process as claimed in claim 2, wherein the organic solvent is selected from acetone, N,N-DMAc, THF, ethyl acetate, ethyleneglycol diethyl ether, 1,2-dimethoxyethane, 1,2-dichloroethane, NMP, DMF, acetonitrile, DMSO, toluene, sulfolane, and ethanol.

4. A process as claimed in claim 1, wherein the amine base is selected from 4-methylmorpholine, triethylamine, 2,6-lutidine, 2,2,6,6-tetramethylpiperidine, N,N′-diethylaniline, DBN, pyridine, diethylamine, and ethanolamine.

5. A process as claimed in claim 1, wherein the salt of Compound 2 is an acetate salt or a hydrochloride salt.

6. A process for preparing Compound 1 as described in claim 1, the process comprising the steps of:
reacting Compound 2 or a salt thereof: 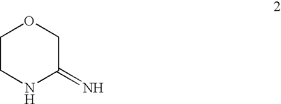
with Compound 10: 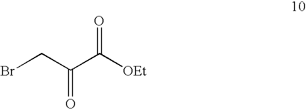
in the presence of an alkali carbonate to yield Compound 11: 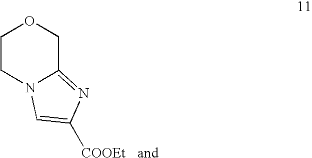
reducing Compound 11 with a reducing agent to yield Compound 1.

7. A process as claimed in claim 6, further in the presence of an organic solvent.

8. A process as claimed in claim 6, wherein the step to yield Compound 11, further comprises dimethoxyethane.

9. A process as claimed in claim 1, wherein Compound 2 or a salt thereof is prepared by the process comprising the steps of:
reacting Compound 4 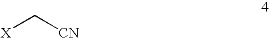
wherein X is Cl, Br, or I;
with Compound 5: 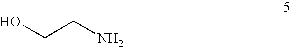
in the presence of a second base to yield Compound 6 or a salt thereof: 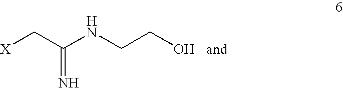
cyclizing Compound 6 or its salt in the presence of a third base to yield Compound 2 or a salt thereof.

10. A process as claimed in claim 9, further in the presence of an organic solvent.

11. A process as claimed in claim 10, wherein the organic solvent is methanol.

12. A process as claimed in claim 9, wherein the third base is potassium t-butoxide, and the cyclization is performed in t-butanol.

13. A process as claimed in claim 9, wherein the salt of compound 6 is the hydrochloride salt.

14. A process as claimed in claim 1, wherein Compound 2 or salt thereof is prepared by the process comprising the steps of:
reacting Compound 4 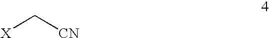
with Compound 5: 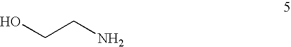
in the presence of a second base to yield Compound 6: 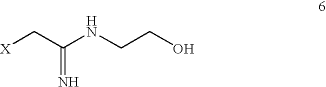
treating Compound 6 with hydrochloric acid to yield a hydrochloride salt, Compound 7: 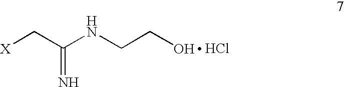
and, cyclizing Compound 7 in the presence of a fourth base to yield Compound 2 or a salt thereof, wherein X is Cl, Br, or I.

15. A process as claimed in claim 14, wherein at least one step is performed in an organic solvent.

16. A process as claimed in claim 1 or 9, wherein Compound 3 is prepared by a process comprising the steps of:
halogenating Compound 8: 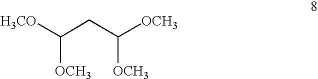
in the presence of a halogenating agent and a first acid to yield Compound 9: 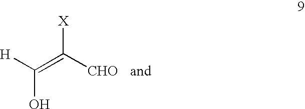
reacting Compound 9 with isopropanol in the presence of a catalytic amount of a second acid to yield Compound 3, wherein X is Cl, Br, or I.

17. A process as claimed in claim 16, wherein the first acid is hydrochloric acid.

18. A process as claimed in claim 16, wherein the final step to yield Compound 3, further comprises refluxing methylcyclohexane or cyclohexane.

19. A process for preparing a compound of formula 12: 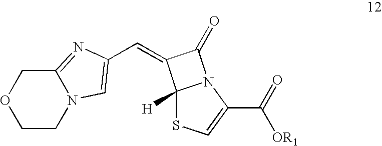
wherein
R₁ is H, a salt selected from Na, K, and Ca, or an in vivo hydrolyzable ester selected from a C₁-C₆ alkyl, a C₅-C₆ cycloalkyl, or a —CHR₂OC(O)C₁-C₆ alkyl; and
R₂ is H, a C₁-C₆ alkyl, a C₃-C₆ cycloalkyl, an optionally substituted aryl, or an optionally substituted heteroaryl,
or a pharmaceutically acceptable salt or hydrate thereof;
the process comprising the steps of:
condensing Compound 1: 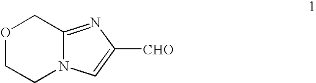
with a 6-bromo-penem derivative of formula 13: 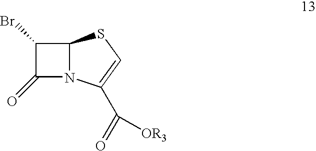
in the presence of a Lewis acid and a fifth-base,
wherein R₃ is para-nitrobenzyl, benzyl, para-methoxy benzyl, benzhydrol, or trityl, to form an intermediate aldol product of formula 14: 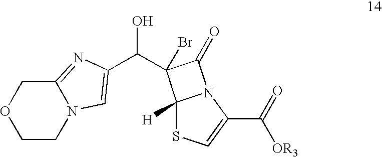
wherein R₃ is as defined above;
reacting the intermediate aldol product of formula 14 with an acid chloride of the formula R₄Cl, an anhydride of the formula (R₄)₂O, or C(X₁)₄ and triphenylphosphine,
wherein R₄ is C₁₋₆alkyl-SO₂—, C₃₋₁₄aryl-SO₂—, C₁₋₆alkyl-C(O)—, or C₃₋₁₄aryl-C(O)—; and X₁ is Br, I, or Cl, to form an intermediate of formula 15: 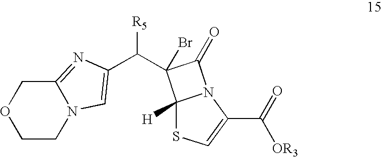
wherein R₅ is —OR₄ or X₁ and R₃, R₄ and X₁ are as defined above; and
converting the intermediate of formula 15 to the compound of formula 12, or a pharmaceutically acceptable salt or hydrate thereof, by a reductive elimination process.

20. A process as claimed in claim 19, wherein the fifth base is an organic base.

21. A process as claimed in claim 20, wherein the organic base is triethylamine, DMAP or diisopropyl ethyl amine.

22. A process as claimed in claim 19, wherein R₅ is acetate, triflate, or tosylate.

23. A process as claimed in claim 19, wherein R₃ is para-nitrobenzyl.

24. A process as claimed in claim 19, wherein the reductive elimination process is carried out using activated zinc and a phosphate buffer at a pH of about 6.5 to 8.0 or by hydrogenation in the presence of a catalyst.

25. A process as claimed in claim 19, further comprising converting the compound of formula 12 to a pharmaceutically acceptable salt, or an in vivo hydrolyzable ester selected from a C₁₋₆alkyl ester, a C₅₋₆cycloalkyl ester, and a —CHR₂OCOC₁₋₆alkyl ester, wherein R₂ is as defined in claim 19.

26. A Compound selected from 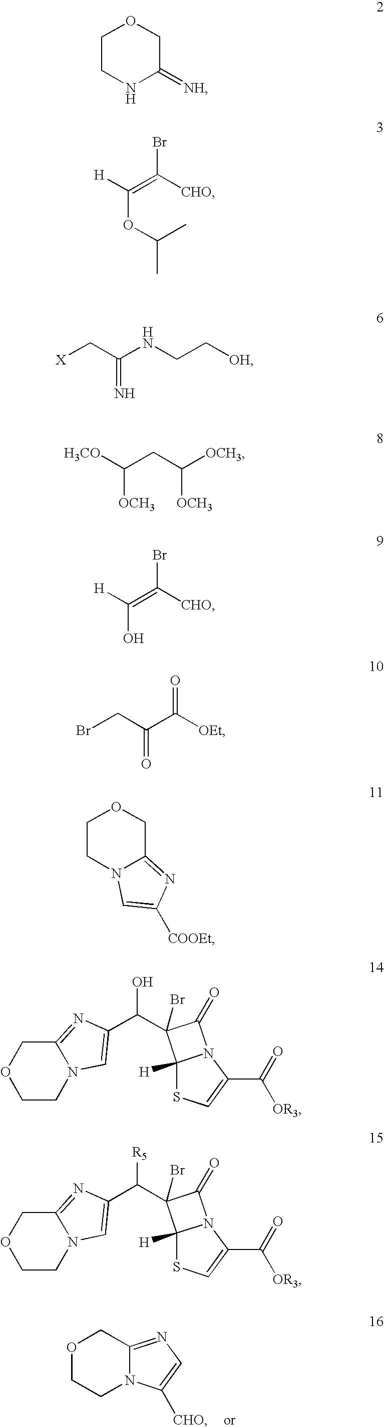 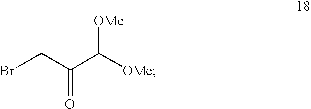
wherein X is Cl, Br, or I;
R₃ is para-nitrobenzyl, benzyl, para-methoxy benzyl, benzhydrol, or trityl;
R₅ is —OR₄ or X₁;
R₄ is C₁-C₆ alkyl-SO₂—, C₁₃₋₁₄aryl-SO₂—, C₁-C₆ alkyl-C(O)—, or C₁₃₋₁₄aryl-C(O)—; and
X₁ is Br, I, or Cl;
or a salt or hydrate thereof.
